(19) United States
(12) Patent Application Publication
(10) Pub. No.: US 2016/0310707 A1
(43) Pub. Date: Oct. 27, 2016

(54) METHOD AND APPARATUS FOR CHILD-BIRTH MONITORING AND ASSISTANCE

(71) Applicant: Kamran Ghodsian, Newport Beach, CA (US)

(72) Inventor: Kamran Ghodsian, Newport Beach, CA (US)

(21) Appl. No.: 14/696,052

(22) Filed: Apr. 24, 2015

(51) Int. Cl. A61M 25/10; A61B 17/42; A61B 5/00; A61B 5/024; A61M 29/02

(52) U.S. Cl. CPC A61M 25/10; A61M 25/10187; A61M 25/10181; A61M 25/1011; A61B 5/02411; A61M 29/02; A61B 5/435; A61B 5/4356; A61B 5/4362; A61M 25/10182; A61B 17/42; A61M 2025/0004

(57) ABSTRACT

A method and an article of manufacture are presented for stimulation of cervical canal, induction of labor, monitoring and control of reproductive system conditions. This specification provides an inflatable system, with one or more balloons that are configured to keep the system in an exact position within woman's reproductive system, monitor pressures inside uterus, control pressures inside uterus, exert controllable pressure on the cervical canal walls, stimulate cervical canal, monitor pressures inside cervical canal, and, if desired, rupture amniotic sac, and the like. In an embodiment a uterine balloon is positioned inside the uterus adjacent to the cervical internal OS; a vaginal balloon is positioned in the vagina for applying pressure on the external cervical OS; and a cervical balloon is positioned in the cervical canal. One or more balloons may be inflated by nontoxic fluids to accurately sense and/or control the intracorporeal pressures by extracorporeal sensors and devices.

### CROSS-REFERENCE(S) TO RELATED APPLICATION(S)

(N/A)

### TECHNICAL FIELD

This application relates generally to processes of child-birth. More specifically, this application relates to methods and apparatus for cervical dilation, labor induction, and monitoring and control of a woman's reproductive system variables.

### BRIEF DESCRIPTION OF THE DRAWINGS

The drawings, when considered in connection with the following description, are presented for the purpose of facilitating an understanding of the subject matter sought to be protected.

FIG. 1 is a schematic diagram of the women's reproductive system during pregnancy;

FIG. 2 schematically illustrates an embodiment of an inflatable system for cervical dilation, labor induction, and uterus and cervical canal pressure monitoring and control.

FIGS. 3A and 3B illustrate examples of possible arrangements for fluid communication from proximate end to the distal end of the inflatable system of FIG. 2; and

FIG. 4 illustrates a sample inflatable system inside a woman's reproductive system; and

FIG. 5 illustrates a sample single-balloon inflatable system inside a woman's reproductive system.

### DETAILED DESCRIPTION

While the present disclosure is described with reference to several illustrative embodiments explained herein, it should be clear that the present disclosure should not be limited to such embodiments. Therefore, the description of the embodiments provided in this specification is illustrative of the present disclosure and should not limit the scope of the disclosure as claimed. In addition, it will be appreciated that the disclosure may include other materials and component arrangements not expressly stated in this specification.

Briefly described, methods and articles of manufacture are disclosed for dilation and stimulation of cervical canal to induce labor, and for measurement, monitoring and control of multiple variables related to a fetus and its surrounding environment. In particular, a special intracorporeal system, comprising balloons, are disclosed to cause cervical dilation and labor induction, while performing monitoring of different aspects and stages of the labor.

There are various reasons to induce labor. For example, when a woman is two or more weeks overdue (postdate), and labor does not start on its own, it may be desirous to induce labor, due to fetal or maternal indications, such as placental dysfunction, pregnancy induced hypertension, Preeclampsia, Diabetes, intra-uterine growth restriction, conditions that may jeopardize fetal well-being, or other conditions that may affect the woman's health.

Labor may be induced by causing the cervix to soften and open. For example, the pharmaceutical substance, prostaglandin (PG), leads to local biochemical and biophysical alterations in the cervical region that reduce cervical resistance and induce myometrial contractions. Endocervical, or vaginal application of PG, in a gel form, is presently used for priming the cervix before labor induction and for labor induction. Currently, the Prepidil Gel is applied by intra-cervical injection using a syringe with a simple canula. However, it is extremely difficult, if not impossible, to administer 3 ml of gel in a strictly endocervical fashion without applying some of the gel retroamniotically, which may cause side effects to uterine hypercontractility, and may lead to fetal distress.

The present disclosure provides an inflatable system, with one, two or three balloons (or more) that are configured to keep the system in an exact position within woman's reproductive system, monitor and/or control pressures inside uterus, exert controllable pressure on the cervical canal walls, stimulate cervical canal, monitor pressures inside cervical canal, and, if desired, administer pharmaceutical substances and/or rupture amniotic sac.

In various embodiments a uterine balloon is positioned at a proximal portion of the uterus, with respect to an operator, adjacent to the cervical internal OS. In some embodiments the uterine balloons may have different shapes so as to, for example, maximize the pressure against the decidua and the internal cervical OS and to minimize the pressure on the fetal head and in other embodiments the uterine balloons may have different surface roughness, for example, for anchoring the uterine balloons in place.

In some embodiments, the inflatable system may have a vaginal balloon for positioning in the vagina for applying pressure on the external cervical OS. The combination of a uterine balloon and a vaginal balloon will stabilize the position of the inflatable system within the woman's reproductive system as long as both balloons are inflated.

In various embodiments, the inflatable system may have a cervical balloon for positioning in the cervical canal, shaped so as to maximize the contact area with the cervix. The shape and the surface roughness of the cervical balloon may also be designed in order to maximize cervix contact and stimulation.

The balloons of the inflatable system are operative partly to stimulate the secretion of hormone by exerting pressure on the proximal decidual surfaces of the uterus and on the cervix, to soften and ripen the cervix, to cause the cervix to dilate, and to induce labor. The balloons, which in some embodiments may have rough external surfaces in order to keep them anchored in place, may be inflated by the operator directly after their insertion, or manually and gradually by the patient. In yet other embodiments, various sensors and instruments may be used as part of the inflatable system to monitor cervical dilation, fetal well-being, and the woman's conditions. In some embodiments, pharmaceutical substances may also be applied to the cervix canal through the cervical canal portion of the disclosed device.

As mentioned above, the presented embodiments disclose an inflatable system that may use a single uterine balloon, a uterine balloon and a vaginal balloon, or a uterine and a vaginal and a cervical balloon, or any combination thereof. In various embodiments, each of the named balloons (vaginal, uterine, cervical) may itself be a combination of several balloons. In some embodiments the uterine balloon and/or the cervical balloon are inflated by incompressible fluids, such as water or oil, to transfer the intracorporeal pressures to extracorporeal pressure sensors. While fluid is the best medium for transfer of pressure throughout the system, air or other gases may also be used for this purpose. In all applications, nontoxic fluid or gases are preferable.

Intracorporeal labor induction systems of the prior art do not control or measure uterus/amniotic-fluid pressure. By clogging the cervical canal, the existing systems make it hard, if not impossible, to insert a separate pressure measuring instrument into a patient's reproductive system to measure uterus and amniotic-fluid pressures. Furthermore, as will be explained below, the available uterus/amniotic-fluid pressure measurement devices are inherently inaccurate. The systems disclosed in this specification can induce labor while measuring the uterus/amniotic-fluid and/or cervical canal pressures very accurately, and even control the uterus and/amniotic-fluid pressure. With the new systems, by applying any desired pressure on the cervix canal walls, the cervical stimulation of the cervix canal can be also controllable.

The balloons, which may have rough external surfaces in order to keep them anchored in place, may be inflated by the operator or by the patient herself. Various sensors and other instruments may be used along with or as a part of the inflatable system to monitor cervical dilation, fetal well-being, and the woman's conditions. The disclosed inflatable system is not limited in its application to the details of construction and the arrangement of the components set forth in this specification or illustrated in the drawings.

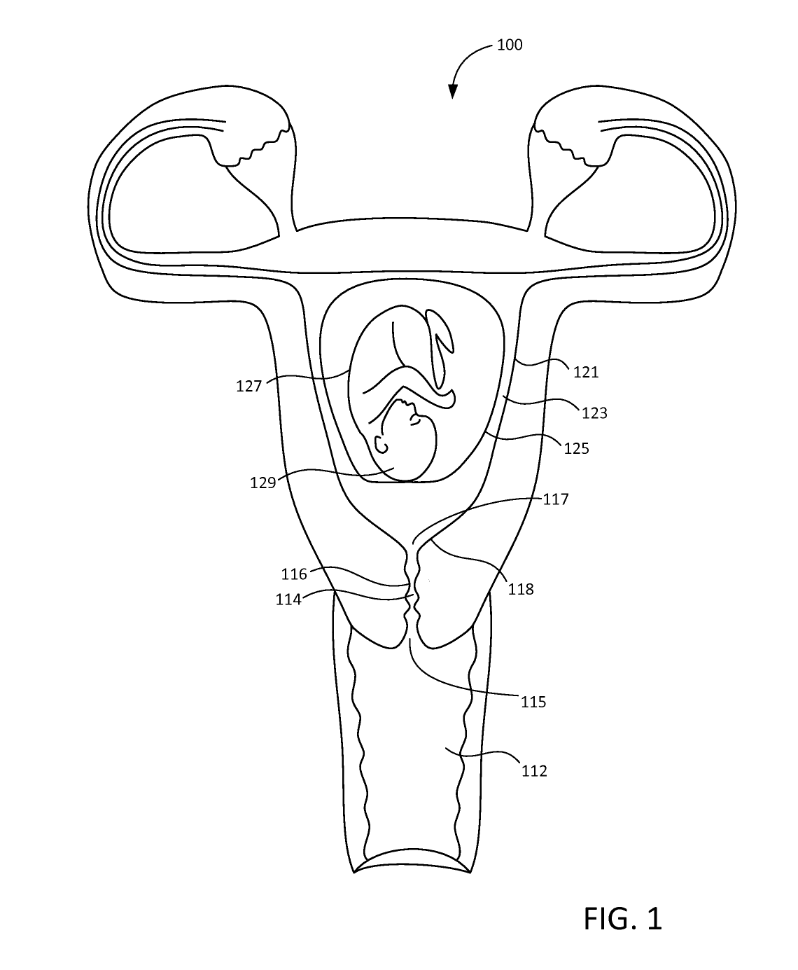

FIG. 1 illustrates a woman's reproductive system 100, during pregnancy, showing a vagina 112, a cervix 116 forming a cervical canal 114, an external cervical OS 115, an internal cervical OS 117, a uterus 118, an endometrium 121 which is the mucous membrane lining the uterus 118, an amniotic sac 125 containing a fetus 127 having a head 129, and a decidua 123 which is the mucous membrane lining the uterus 118 in preparation for, and during pregnancy.

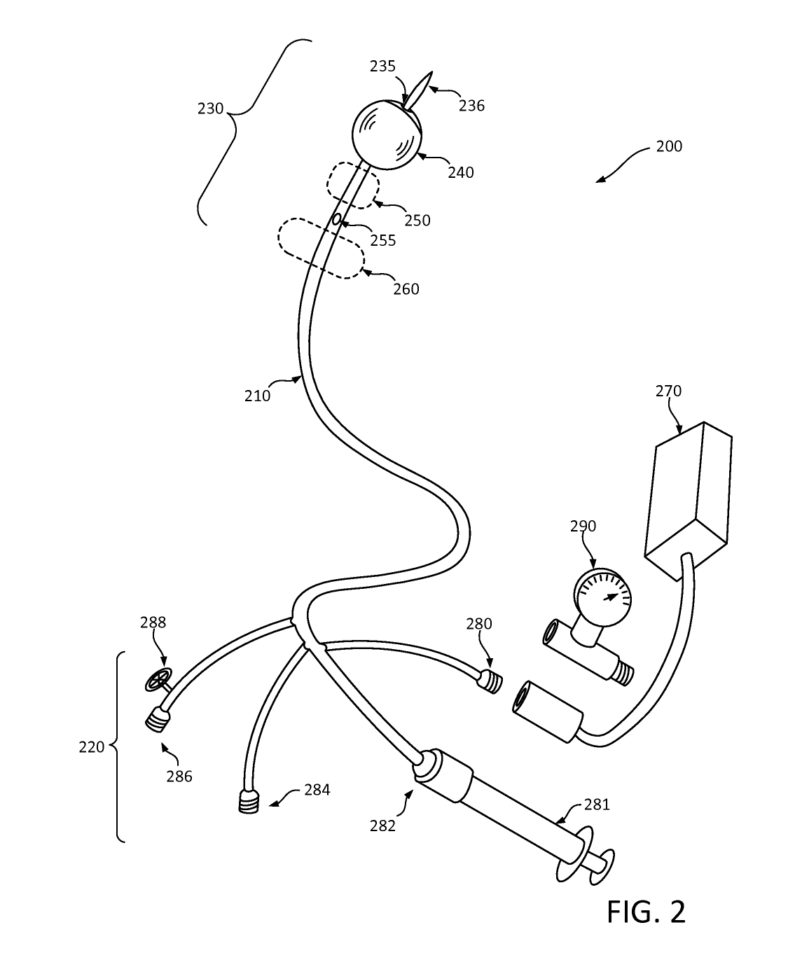

FIG. 2 schematically illustrates an embodiment of the inflatable system 200 for cervical dilation, labor induction and uterus and cervical canal pressure monitoring and control. As seen in FIG. 2, the inflatable system 200 includes a catheter 210 having a proximal end 220 and a distal end 230, with respect to an operator, wherein the distal end 230 enters a woman's reproductive system and the proximal end 220 remains outside her body to be manipulated by an operator/medical staff. The catheter 210 includes: (1) a uterine balloon 240 substantially at the distal end 230 and a conduit/tube (not shown) inside the catheter 210, spanning from the proximal end 220 to the distal end 230 and in fluid communication with the uterine balloon 240.

In various embodiments, the catheter 210 also includes a cervical balloon 250, in fluid communication with a cervical-balloon conduit/tube (not shown) inside the catheter 210, spanning from the proximal end 220 to the distal end 230. As seen in FIG. 2, the location of the cervical balloon 250 on the catheter 210 is closer to catheter's proximal end 220 than the location of the uterine balloon 240 is. The catheter 210 is designed for insertion into the woman's reproductive system, so that the cervical balloon 250 is positioned and inflated within the cervical canal 114. The cervical balloon 250 is designed for applying pressure on the cervix 116. It will be appreciated that the pressure on the cervix 116 may further include pressure on the internal cervical OS 117 and on the external cervical OS 115.

In some embodiments, the inflatable system 200 may include a vaginal balloon 260, in fluid communication with a vaginal-balloon conduit/tube (not shown) inside the catheter 210, spanning from the proximal end 220 to the distal end 230. The vaginal balloon 260 is adapted for positioning within the vagina 112, at a distal portion thereof, for pressing against the external cervical OS 115. The one, two or three-balloon inflatable system 200, from its distal end 230, is inserted into a woman's reproductive system 100, prior to inflation of the balloons.

In an exemplary three-balloon inflatable system 200, the uterine balloon 240 and the vaginal balloon 260 are partly operative to anchor the cervical balloon 250 in place and to enhance hormonal secretion by applying pressure on the decidual 123, the internal cervical OS 117, and the external cervical OS 115. At the same time, the cervical balloon 250, anchored within the cervical canal 114, may accelerate cervical dilation, by applying pressure on the cervix 116, and preferably also, the internal cervical OS 117 and on the external cervical OS 115.

In various embodiments the inflatable system 200 may also have openings, such as opening 255, which is in fluid communication with a conduit/tube (not shown) inside the catheter 210, spanning from the proximal end 220 to the distal end 230. Opening 255 is situated on the catheter 210 such that, for example, upon placement in a woman's reproductive system the opening 255 is positioned within the cervical canal 114 and is used to insert pharmaceutical substances into the cervical canal 114.

In accordance with the present invention, the inflation of the balloons of the inflatable system 200 may be performed by a doctor or a midwife (not shown) directly after its insertion. Alternatively, the inflation of the balloons of the inflatable system 200 may be performed by the patient via a hand pump 270, in a gradual manner, for example over several hours. Alternatively still, uterine balloon 240 and vagina balloon 260 may be inflated by the doctor or midwife, while the inflation of the cervical balloon 250 may be performed by the patient, for example, via a hand pump 270. (Pump 270 in FIG. 2, represents any kind of manual or automatic gas or fluid pump.)

Having a pressure sensor at the distal end of the inflatable instrument, inside the uterus and/or cervix canal, has some limitations and disadvantages including: (1) the pressure sensor must be small enough, such as a few millimeters, to fit and/or move inside the instrument, which has to pass through the cervix canal; (2) the pressure sensor must be disposable since the entire intracorporeal part of the system needs to be discarded after use; (3) the sensed pressures need to be transmitted outside the woman's body for recording or display, by wire or wirelessly; and (4) the system will be more expensive and bulkier as a result of the mentioned limitations and disadvantages.

An inflatable system for labor induction and monitoring/control will be much smaller, less expensive and simpler if the pressures inside the uterus and/or cervical canal can be transferred to a sensor(s) situated outside the woman's body. To do so, a very simple and instantaneous transfer of pressure is possible by using gasses and fluids. However, pressure transfer through a gaseous medium is not as accurate as through incompressible fluids since gasses are compressible and their compressibility varies by temperature and pressure, which introduce undesirable variables into the sensing subsystem. On the other hand, pressure may be accurately sensed at any point of a fluid body without any erroneous effect from the surrounding variables. Therefore, the preferred embodiment of this specification inflates, at least, the uterine balloon by nontoxic incompressible fluids such as water.

In the uterus, as a result of hydraulic principles, the pressure inside the fluid-filled uterine balloon will rapidly come to static equilibrium with the pressures of the amniotic sac and the uterus walls. At such stage, the pressure of the amniotic fluid, the uterine balloon fluid and the pressure on the uterus walls are equal. Hence a sensor outside the woman's body and in fluid contact with the uterine balloon will sense the exact pressure of the uterus/amniotic fluid. The uterus/amniotic fluid pressure may be controllably increased or decreased by inflating or deflating the uterine balloon, respectively. While inflating or deflating the uterine balloon, the exact pressure inside the uterus may also be sensed and observed. With fluid, no wait time is required for pressure equilibrium.

The same is true about a cervical balloon and the monitoring and control of the cervix pressures. In some embodiments, the fluid inside the cervical canal balloon may even be pulsated to further stimulate the canal walls. The pulsation may be caused manually or mechanically. Here also, the cervical balloon itself may be a combination of multiple balloons which may be inflated separately or as a group.

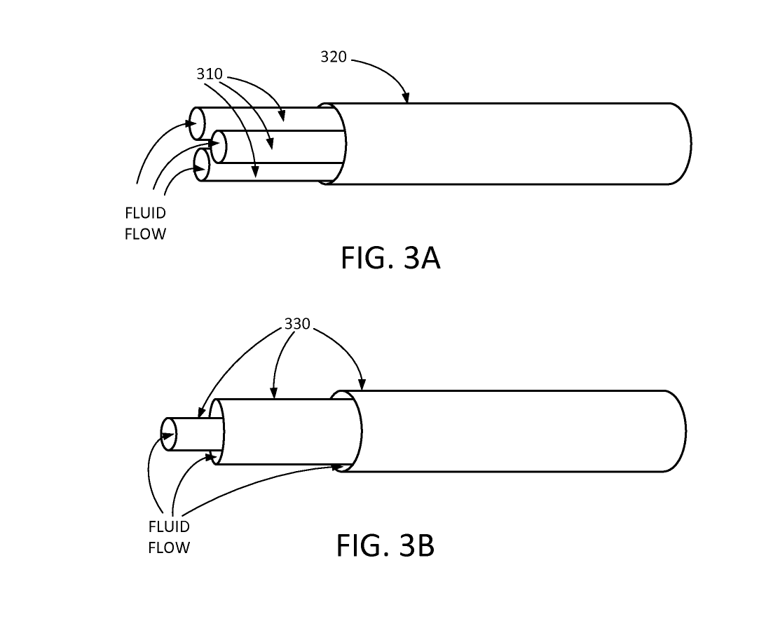

The inflation/deflation of multiple balloons may be controlled together or independently to allow stimulation of the cervical canal with variable pressures across both time and position within the cervical canal. Such an arrangement may be used to create a pressure signal, varying over time and space, to stimulate different regions within the canal at different pressures and at different times, which may be helpful in stimulating dilation more effectively. For example, if two balloons are employed, their inflation and deflation may be alternated to create a two-point variable pressure signal. In some embodiments, the inflatable system 200 may be coupled with a pressure controller to automatically inflate and deflated the multiple balloons according to a predefined signal pattern chosen by the operator. For example, the pressure controller may have several settings such as sinusoidal pattern, ramped inflation/deflation, and pulsed/rapid inflation/deflation, each of which may be selected by the operator to inflate and deflate the balloons accordingly for different types of stimulation. In multiple balloon embodiments, each balloon may need its own separate micro-tube for independent inflation/deflation, in which the micro-tubes pass through a larger outer tube to allow easy handling and control of the inflatable system, in a manner similar to those shown in FIGS. 3A and 3B discussed below. In alternate embodiments, each of the multiple balloons used in cervical canal stimulation may be equipped with an electronically controlled micro-valve to open to allow fluid in and out to the extent desired and then closed again to isolate the balloon.

In general, the vaginal balloon need not be inflated by fluid because it is usually not used to sense or control any pressure; however, if fluid is used to inflate the other balloons, it may be easier to do the same with the vaginal balloon. The vaginal balloon may also consist of more than one balloon controlled as a group or separately in a manner similar to the cervical canal balloons described above.

In preferred embodiments at least one of the balloons is inflated by fluid such as water to accurately measure and control the pressure of its surrounding. An increase or decrease of the size of the balloons will increase or decrease, respectively, the pressure of their environment which at the same time can be measured. For example a small fluid inflation of the uterine balloon 240 enables an accurate measurement of the uterus and amniotic fluid pressures while any increase in the uterine balloon size will increase the uterus and amniotic fluid pressures. Same is true with cervical canal pressure measurement and control. Usually there is no need for measurement and/or control of vaginal pressure and therefore the vaginal balloon may be inflated by air instead of a fluid.

FIG. 2, shows an example system 200 with four connection ports 280, 282, 284, and 286, to which gas and fluid pumps, sensors and gauges, and other instruments may be attached. Each port may be of different kind and use different operating mechanisms. For example connection port 280 may be closed in its unattached state and be open as long as being connected to any device. Or, for example, port 286 may have a manually operated valve 288.

Therefore, connection port 280, which in this example is assumed to be in fluid communication with uterine balloon 240, may first be connected to a pressure gauge 290 and a fluid pump 270 until the uterine balloon 240 is inflated to a desired pressure or size and subsequently be disconnected from the pump, if needed. In such a case, the uterus pressures can be continuously monitored. If constant monitoring of the pressure is not needed, both the pressure gauge 290 and the fluid pump 270 may be removed and the pressure of uterus be checked at the connection port 280 whenever desired. In some embodiments, the conduit/tube connecting connection port 280 to uterine balloon 240 may even have a separate dedicated port for connecting to sensors and gauges. In yet another example, connection port 280 may be attached to an automated machine for monitoring and/or controlling the pressure of the uterus. In various embodiments, dedicated monitoring ports for each balloon may be connected to a monitoring device(s) while ports, such as port 280, are dedicated to injection, manipulation and/or pumping devices. In these embodiments the pumping device can be a mere syringe 281. There is no need for complicated pumping devices with pressure gauge.

Assuming that connection port 284 is in fluid communication with cervical balloon 250, it can be connected to a fluid pulsating device that is capable of controlled fluid pulsation with desired low and high pressures.

If fluid is used to inflate the balloons, there are several ways to eliminate the air in the conduits/tubes. In one embodiment the conduits and the balloons of the system 200 may be manufactured in a collapsed or vacuumed initial form. In other embodiments the connection ports may provide manual release valves to empty the trapped air while inserting fluid into the conduits. Such solutions are known to those skilled in the art and need no further elaboration.

As illustrated in FIG. 2, different devices may be used as a part of the inflatable system 200. In various embodiments the inflatable system 200 may include at least one additional conduit/tube (not shown), having at least one opening 235, to the uterus. The at least one additional conduit/tube is operable for inserting at least one device 236 to the woman's reproductive system. In the embodiment of FIG. 2, the at least one additional tube is operable for inserting the at least one device 236 into the uterus, via the opening 235. Similarly, another conduit/tube may be used for inserting another device into the cervical canal, via a similar opening. In FIG. 2, the at least one device 236 is a device for rupturing the amniotic sac. Device 236 may be operated as a plunger-like device, using a handle at the proximal end 220 of the inflatable system 200. After the puncture, device 236 may be withdrawn from one of the connection ports.

In yet other embodiments, the at least one device 236 may be a sensor, for example, a for sensing amniotic-fluid temperature, or for sensing fetal heart-beat, preferably operable via the opening 235 to the uterus. The measurements may be transmitted extracorporeally in a wired or wireless manner.

It will be appreciated that the device 236 may be selected from the group consisting of device for breaking water, a device for sensing amniotic-fluid temperature, a device for sensing fetal heart-beat, a device for measuring an extent of cervical dilation, a device for measuring a frequency of uterine contractility, a device for measuring an intensity of uterine contractility, a device for amnioscopy, a device for fetoscopy, a device for scalp blood pH sampling, and the like.

Alternatively, the at least one device 236 may include a drug form, designed for passive dispensing of a medication. The drug form may be inserted with a syringe 281, and the syringe 281 may then be withdrawn. The passive dispensing of a medication may be by instantaneous release, delayed release, pulsating release, timed release, slow release, or another release form, as known, operable via the opening 235 to the uterus, or via a similar opening(s) to the cervical canal. Device 236 may be electronically-controlled, pre-programmed, or may be wirelessly controlled from an extracorporeal station.

In yet other embodiments, device 236 may include a device for measuring an extent of cervical dilation, operable via an opening to the cervical canal. The measurements may be transmitted extracorporeally by wire or wirelessly. In various embodiments, device 236 may be a combination of several devises and/or sensors.

FIG. 3A and 3B illustrate two example possible arrangement of vaginal, cervical and uterine conduits/tubes for fluid communication with vaginal, cervical and uterine balloons, respectively, and alternatively with openings such as cervical opening 255. In FIG. 3A, separate tubes 310 are within a conduit 320 and each connect a connection port to a balloon or an opening. In FIG. 3B, the tubes 330 are nested without a need for a conduit. In other embodiments the arrangement of the tubes may be a combination of the arrangements in FIGS. 3A and 3B.

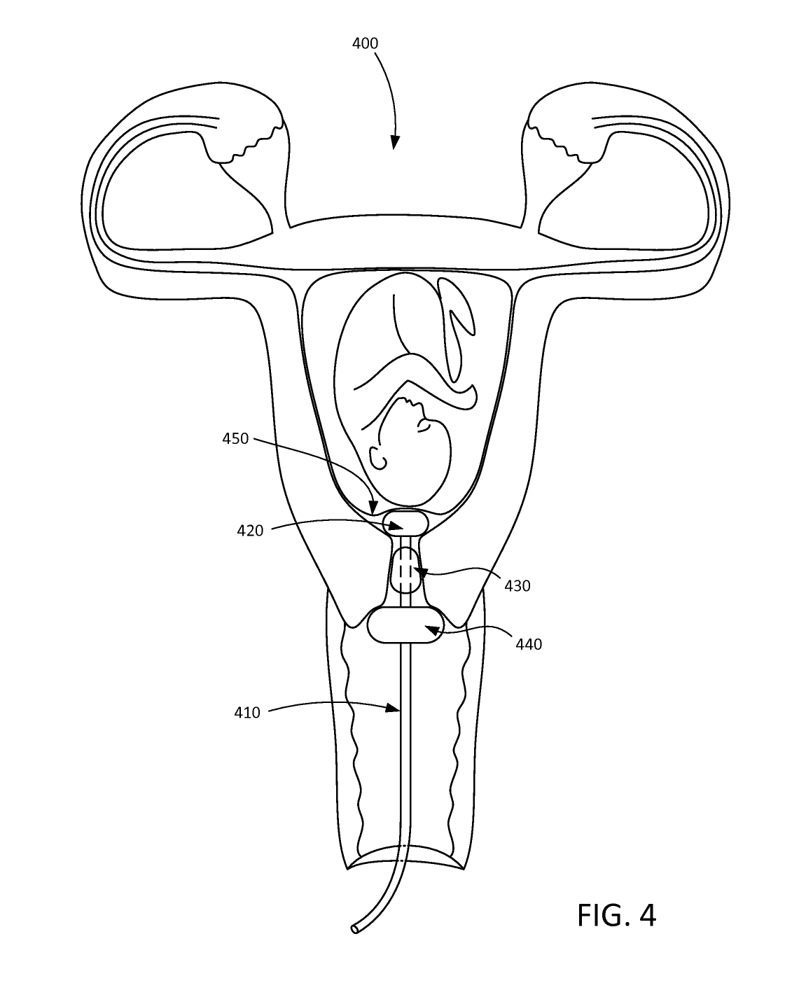

As shown in FIG. 4, the three-balloon catheter 410 is inserted into a woman's reproductive system 400 and subsequently inflated, so that the uterine balloon 420 is in contact with the amniotic sac 450 in the lower segment of the uterus, the cervical balloon 430 is in the cervical canal, and the vaginal balloon 440 is in the distal end of the vagina. In the example of FIG. 4, the uterine balloon 420 applies pressure on the decidua and the internal cervical OS. The cervical balloon 430 applies pressure on the cervix walls, and the vaginal balloon 440 applies pressure on the external cervical OS. For withdrawal, the balloons are deflated.

As seen from FIG. 4, the uterine balloon 420 is operative partly to separate the amniotic sac 450 from the decidua in the lower segment of the uterus, thereby stimulating endogenous hormone secretion of PG from the decidua. The hormone secretion by the decidua is operative to soften and ripen the cervical canal and induce labor. Similarly, the cervical balloon 430 is operative to stimulate hormone secretion by the cervix. Again, the hormone secretion is operative to soften and ripen the cervical canal and induce labor.

In some embodiments electrocardiogram of the fetus heart may be obtained by temporarily or permanently attaching a desired type and number of electrodes on the surface of the uterine balloon 420, to be directly in contact with the fetus head or indirectly through the amniotic sac, and to send the sensed signals to an extracorporeal EKG or ECG machine, by wire or wirelessly. In some embodiments the electrodes may be a part of or be ingrained in the material of the uterine balloon 420.

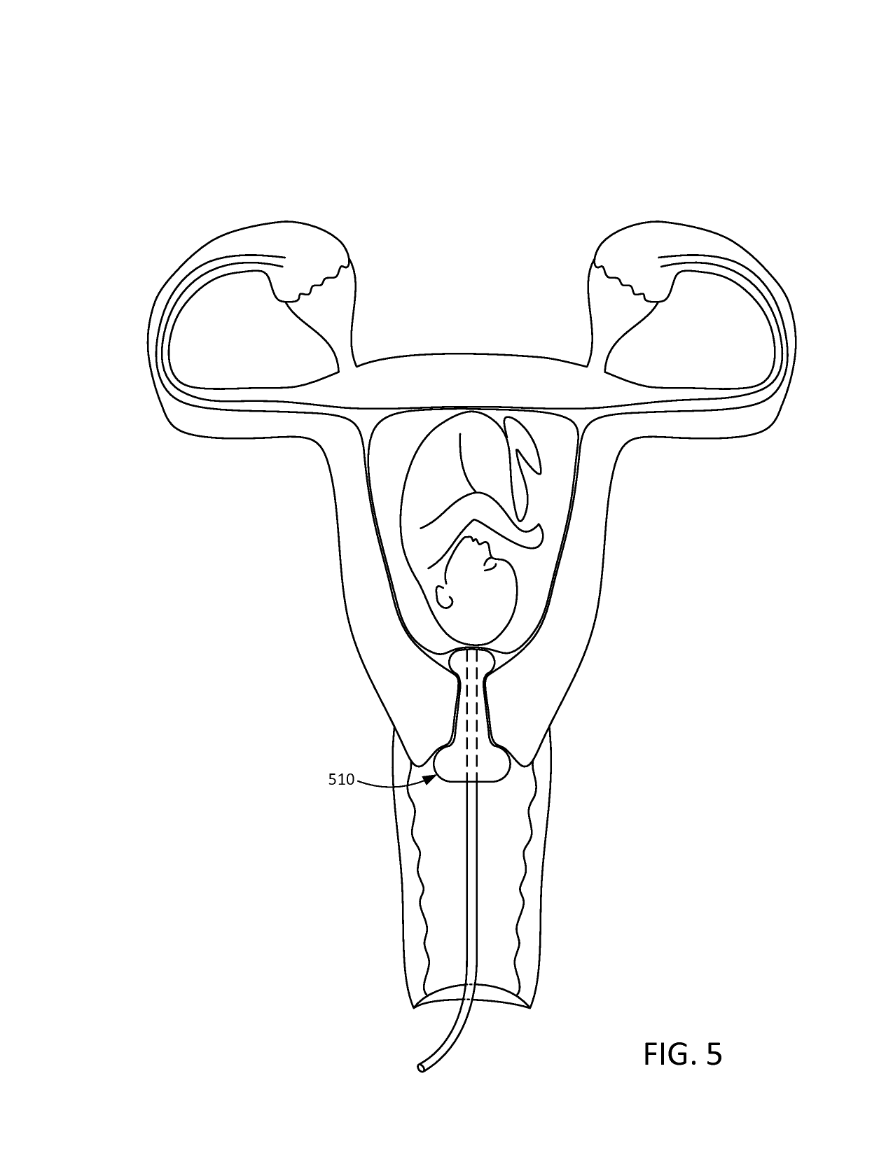

As mentioned above and illustrated in FIG. 5, the inflatable system balloons may have different shapes and may replace other balloons. In the example embodiment of FIG. 5, a single balloon 510 has replaced vaginal, cervical, and uterine balloons.

Changes can be made to the claimed invention in light of the above Detailed Description. While the above description details certain embodiments of the invention and describes the best mode contemplated, no matter how detailed the above appears in text, the claimed invention can be practiced in many ways. Details of the system may vary considerably in its implementation details, while still being encompassed by the claimed invention disclosed herein.

Particular terminology used when describing certain features or aspects of the invention should not be taken to imply that the terminology is being redefined herein to be restricted to any specific characteristics, features, or aspects of the invention with which that terminology is associated. In general, the terms used in the following claims should not be construed to limit the claimed invention to the specific embodiments disclosed in the specification, unless the above Detailed Description section explicitly defines such terms. Accordingly, the actual scope of the claimed invention encompasses not only the disclosed embodiments, but also all equivalent ways of practicing or implementing the claimed invention.

The above specification, examples, and data provide a complete description of the manufacture and use of the composition of the invention. Since many embodiments of the invention can be made without departing from the spirit and scope of the invention, the invention resides in the claims hereinafter appended. It is further understood that this disclosure is not limited to the disclosed embodiments, but is intended to cover various arrangements included within the spirit and scope of the broadest interpretation so as to encompass all such modifications and equivalent arrangements.

All publications, patents and patent applications mentioned in this specification are herein incorporated in their entirety by reference into the specification, to the same extent as if each individual publication, patent or patent application was specifically and individually indicated to be incorporated herein by reference. In addition, a citation or identification of any reference in this application shall not be construed as an admission that such reference is available as prior art to the present invention.

While the present disclosure has been described in connection with what is considered the most practical and preferred embodiment, it is understood that this disclosure is not limited to the disclosed embodiments, but is intended to cover various arrangements included within the spirit and scope of the broadest interpretation so as to encompass all such modifications and equivalent arrangements.

1. An inflatable apparatus for cervical dilation, labor induction and pressure monitoring and pressure control of a woman's reproductive system, the apparatus comprising:
a catheter with a distal end constructed to be placed inside the woman's reproductive system and a proximal end constructed to be kept outside a woman's body, configured for manipulation by an operator;
at least one balloon situated substantially at the distal end of the catheter, configured to be placed at least partially inside woman's uterus;
at least one electrode and/or one device attached to a surface of the balloon, temporarily or permanently, for electrocardiogram of a fetus heart, sensing amniotic-fluid temperature, fetal heart-beat, measuring an extent of cervical dilation, measuring a frequency of uterine contractility, measuring an intensity of uterine contractility, aminioscopy, fetoscopy, scalp blood pH sampling, or any combination thereof;
a conduit extending from the proximal end to the balloon, through which the balloon is inflated with a gas or a fluid;
a port for connecting a pressure sensing device to the conduit at the proximal end of the catheter to allow observing and/or recording a pressure transferred by the gas or the fluid from the balloon to the pressure sensing device; and
an optional port to allow connecting a pumping device to the conduit at the proximal end of the catheter to allow inflating or deflating the balloon.

2. The apparatus of claim 1, wherein the apparatus is configured to send measured and/or sensed signals to an extracorporeal machine by wire or wirelessly.

3. The apparatus of claim 1, further comprising one more balloon, wherein one balloon is configured to be placed inside a woman's uterus and the other balloon is configured to be placed inside a woman's vagina or cervical canal.

4. The apparatus of claim 1, further comprising two more balloons, wherein one balloon is configured to be placed inside a woman's uterus, one balloon is configured to be placed inside a woman's vagina and one balloon is configured to be placed inside a woman's cervical canal.

5. The apparatus of claim 1, wherein the electrode and/or the device is a part of or is ingrained in a material of the balloon.

6. The apparatus of claim 5, wherein the fluid is water or another nontoxic fluid.

7. The apparatus of claim 2, wherein, to obtain electrocardiogram of the fetus heart, the electrode is configured to be directly in contact with the fetus head, or is configured to be indirectly in contact through the amniotic sac, and wherein the apparatus is configured to send sensed signals to an extracorporeal EKG or ECG machine by wire or wirelessly.

8. The apparatus of claim 1, further comprising an opening in the catheter, close to the distal end, and a conduit extending from the proximal end to the opening, through which conduit a pharmaceutical substance is introduced into the woman's reproductive system.

9. The apparatus of claim 1, further comprising an opening in the catheter, close to the distal end, and a conduit extending from the proximal end to the opening, through which conduit an instrument is inserted into the woman's reproductive system.

10. The apparatus of claim 1, wherein the apparatus is equipped for sensing fetal heart-beat, measuring frequency of uterine contractility, and measuring intensity of uterine contractility.

11. The apparatus of claim 1, wherein the apparatus includes the optional pumping port and the pumping device is manual or automatic.

12. The apparatus of claim 1, wherein the conduits are concentric, non-concentric or a combination of concentric and non-concentric.

13. The apparatus of claim 1, wherein the apparatus includes the optional pumping port and the pumping device is a syringe.

14. The apparatus of claim 1, wherein the electrode and/or the device is a part of a material of the balloon.

15. A method of cervical dilation, labor induction and pressure monitoring and pressure control of a woman's reproductive system, the method comprising:
inserting a catheter into the woman's reproductive system through her vagina and cervical canal, wherein the catheter has a distal end placed inside the woman's reproductive system and a proximal end kept outside woman's body and wherein the catheter has at least one balloon attached at or near the distal end, configured to be inserted into woman's uterus, and the balloon is in fluid communication with the proximal end;
attaching at least one electrode and/or one device to a surface of the balloon, temporarily or permanently, for electrocardiogram of a fetus heart, sensing amniotic-fluid temperature, fetal heart-beat, measuring an extent of cervical dilation, measuring a frequency of uterine contractility, measuring an intensity of uterine contractility, amnioscopy, fetoscopy, scalp blood pH sampling, or any combination thereof
inflating the balloon from the proximal end and through the catheter by a fluid or a gas;
measuring the gas or fluid pressure at the proximal end of the catheter; and
optionally controlling the pressure of balloon's surrounding by further inflating or deflating the balloon.

16. The method of claim 15, wherein the balloon is inserted into woman's uterus.

17. The method of claim 15, further comprising one more balloon, wherein one balloon is placed inside woman's uterus and the other balloon is placed inside woman's vagina or cervical canal.

18. The method of claim 15, further comprising two more balloons, wherein one balloon is placed inside woman's uterus, one balloon is placed inside woman's vagina and one balloon is placed inside woman's cervical canal.

19. The method of claim 15, wherein the balloon is inflated manually or automatically.

20. The apparatus of claim 15, wherein the balloon is in fluid communication with the proximal end of the catheter through a tube inside the catheter.
